(19) United States
(12) Patent Application Publication
(10) Pub. No.: US 2005/0271606 A1
(43) Pub. Date: Dec. 8, 2005

(54) Composition for treating inflammation which contains zinc pyrrolidonecarboxylate

(76) Inventors: Keiji Iwasaki, Kawasaki-shi (JP); Manabu Kitazawa, Kawasaki-shi (JP); Yoshinobu Takino, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/146,111

(22) Filed: Jun. 7, 2005

(30) Foreign Application Priority Data
Jun. 8, 2004 (JP) 2004-169655

(51) Int. Cl. A61K 31/555; A61K 7/42

(52) U.S. Cl. 424/59; 514/184

(57) ABSTRACT

The present invention provides a composition which suppresses inflammation of the skin. The composition of the present invention contains zinc pyrrolidonecarboxylate. The composition of the present invention may be formulated as a cosmetic or any externally applied agent.

### BACKGROUND OF THE INVENTION

1. Field of the Invention

The present invention relates to a composition for treating inflammation which is useful for prevention, retardation, and/or improvement of skin diseases and skin damage caused by inflammation.

### BRIEF DESCRIPTION OF THE RELATED ART

Many factors cause inflammation of the skin, including infectious substances such as bacteria and viruses, antigenic substances which cause allergic reactions, tissue-damaging factors, and ultraviolet rays which cause skin cancer or accelerate aging of the skin. Due to such factors and the resulting inflammation, the skin can be damaged in various ways. However, suppressing inflammation reactions can result in the reduction of damage to the skin.

In recent years, studies concerning the mechanism of action of skin inflammation have progressed and, as to causes therefor, the participation of inflammatory cytokines such as IL-1α and TNFα, or an extracellular matrix degrading enzyme such as collagenase has been clarified. The expression of cytokines and extracellular matrix degrading enzymes is controlled on the genetic level by transcription control factors such as NF-κB and AP-1. It has been reported, for example, that when the skin is exposed to the ultraviolet rays contained in sunlight, NF-κF and AP-1 in skin cells are activated and result in the acceleration of aging of the skin (e.g., Nature, Volume 379, pages 335-339, 1996). Accordingly, if these inflammatory factors can be suppressed, skin damage caused by various factors is expected to be mitigated.

Various substances which suppress these inflammatory factors have been reported. For example, it has been shown that an antioxidant substance such as N-acetyl-L-cysteine suppresses activation of NF-κB and AP-1 in epidermal cells (e.g., Free Rad. Biol. Med., Volume 26, pages 174-183, 1999 and FEBS Letters, Volume 384, pages 92-96, 1996). However, the effective concentration is 10 mM to 30 mM, and often the effect is insufficient or highly toxic to the cells. Besides N-acetyl-L-cysteine, retinoic acid has been reported to suppress AP-1 and expression of extracellular matrix degrading enzymes (e.g., Nature, Volume 379, pages 335-339, 1996). However, the side effects of retinoic acid include irritation and skin detachment, and therefore, its use is limited. Zinc salts of amino acids and zinc salts of fatty acids are known to suppress skin damage caused by ultraviolet rays, due to the induction of metallothionein, which is an endogenous antioxidant substance (e.g., WO 00/44341 and WO 93/14748). However, these compounds are not very soluble in aqueous or oily substances, so they are not often used in cosmetics and other agents for external application to the skin, since their effect is often insufficient. In addition, there have been no reports that these substances suppress the activation of a gene transcription factor such as AP-1. Zinc oxide has been reported to suppress diaper rash (e.g., Eur. Acad. Dermatol. Veneol., 15 (Suppl. 1) pages 5-11, 2001), but is also sparingly soluble in aqueous and oily substances; and therefore, it is not usually used in cosmetics and other agents for external application to the skin and, in addition, the effect is insufficient. On the other hand, a water-soluble zinc salt such as zinc acetate has been reported to possibly prevent arteriosclerosis, due to suppression of AP-1 activity. AP-1 activity increases during incubation of hemoendothelial cells under zinc-deficient conditions (J. Am. Clloge Nutrt., 16 (5), pages 411-417, 1997), although it is not known to suppress skin inflammation.

### SUMMARY OF THE INVENTION

An object of the present invention is to provide a composition for suppressing inflammation, wherein said composition is able to prevent, improve, or treat inflammatory disorders by suppressing the activation of a gene transcription factor of extracellular matrix degradation enzymes.

In order to achieve the above-mentioned object, the present inventors have conducted extensive studies and found that a water-soluble zinc salt such as zinc pyrrolidonecarboxylate suppresses the activation of a gene transcription factor of extracellular matrix degrading enzymes.

Thus, it is an object of the present invention to provide a composition for treating inflammation comprising zinc pyrrolidonecarboxylate.

It is a further object of the present invention to provide a composition for treating skin inflammation comprising zinc pyrrolidonecarboxylate.

It is a further object of the present invention to provide the composition as described above, wherein zinc pyrrolidonecarboxylate is either in the L- or DL-configuration.

It is a further object of the present invention to provide the composition as described above which is a selected from the group consisting of an oil, lotion, cream, foundation, gel, shampoo, hair rinse, hair conditioner, enamel, lipstick, face powder, ointment, tablet, injection, granule, capsule, perfume, powder, cologne, toothpaste, soap, cleansing foam, bath salt, hair tonic, and sunscreen.

It is a further object of the present invention to provide a method for treating skin which has been exposed to ultraviolet rays comprising administering the composition as described above.

It is a further object of the present invention to provide a method for treating skin inflammation comprising administering the composition as described above.

It is a further object of the present invention to provide a method for preventing aging of the skin comprising administering the composition as described above.

It is a further object of the present invention to provide a method for preventing or treating hypersensitivity to sunlight comprising administering the composition as described above.

It is a further object of the present invention to provide the method as described above, wherein said hypersensitivity to sunlight is caused by xeroderma pigmentosum or solar urticaria.

It is a further object of the present invention to provide a method for preventing or treating a light allergy comprising administering the composition as described above.

It is a further object of the present invention to provide a method for preventing or treating optical immunosuppression comprising administering the composition as described above.

It is a further object of the present invention to provide a method of preventing or treating chapping of the skin comprising administering the composition as described above.

It is a further object of the present invention to provide the method as described above, wherein said chapping is caused by a wound or cracking of the skin.

It is a further object of the present invention to provide a method of preventing or treating skin diseases comprising administering the composition as described above.

It is a further object of the present invention to provide method as described above, wherein said disease is selected from the group consisting of acne, keratosis, xeroderma, ichthyosis, and psoriasis.

### DESCRIPTION OF THE PREFERRED EMBODIMENTS

In accordance with the present invention, a composition for suppressing inflammation which is advantageously able to be used in cosmetics and other agents is described.

Specific examples of the compound of the present invention will be illustrated. Zinc pyrrolidonecarboxylate (hereinafter, referred to as “PCA zinc salt”) is encompassed by the present invention, and may be in the form of L-PCA zinc salt or DL-PCA zinc salt. Each may be used alone or mixed together and, when a DL-compound is used, there is no particular limitation for the ratio of D-moiety to L-moiety. When PCA zinc salt of the present invention is formulated as a cosmetic or agent for external application to the skin for prevention and improvement of inflammatory skin disease and inflammatory skin damage, its compounding amount may be 0.01% to 10% by weight or, preferably, 0.1% to 5% by weight. When the amount is less than 0.01% by weight, suppression of inflammation is poor; while, when the amount is more than 10% by weight, a squeaking sound can result when the skin is touched, making these amounts undesirable. When PCA zinc salt is formulated as an agent for external application to the skin, it is possible to add additional excipients which are commonly used in cosmetics or other agents for external application to the skin, as long as such excipients do not inhibit the action of the composition of the present invention.

Such commonly used excipients include antioxidants, anti-inflammatory agents, ultraviolet absorbers, whitening agents, cell activators, humectants, metal chelating agents, oily materials, surfactants, solvents, macromolecular substances, powdery substances, dyes, perfumes, accelerators for percutaneous absorption, and steroid hormones.

The dosage form for the cosmetic or agent for external application to the skin is not particularly limited, as long as it contains the above-mentioned PCA zinc salt, and may include a solution, paste, gel, solid, or powder. Cosmetics or agents for external application to the skin according to the present invention may also be in the form of an oil, lotion, cream, milky lotion, gel, shampoo, hair rinse, hair conditioner, enamel, foundation, lipstick, face powder, pack, ointment, tablet, injection, granule, capsule, perfume, powder, cologne water, tooth paste, soap, aerosol, and cleansing foam. The composition of the present invention may also be formulated as a preventative agent for skin aging, a preventative agent for skin inflammation, a bath salt, hair tonic, beauty lotion for the skin, preventative agent for sunburn, preventative agent for hypersensitivity to sunlight, such as xeroderma pigmentosum and solar urticaria, a preventative agent for a light allergy, and a preventative agent for optical immunosuppression, or a preventative agent for skin chapping caused by external wound, chaps, cracks, etc., a disinfectant, antibacterial agent, insecticide, exterminator for noxious insects, a keratolytic agent, epidermis exfoliating agent, preventative agent for pimples, and a preventative agent for various skin diseases such as keratosis, xeroderma, ichthyosis, and psoriasis.

Other common components in cosmetics or agents for external application to the skin may also be added to the composition of the present invention to the extent that such component does not reduce the efficiency of the present invention. Examples of other common components include antiseptic, fading preventer, buffer, drug for pimple, preventative agent for dandruff and itching, antiperspirant/deodorant, drugs for thermal injury, agent against mites and lice, keratin softener, drug for xeroderma, antiviral agent, hormone, vitamin, amino acid/peptide, astringent agent, refreshing/stimulating agent, component derived from animals or vegetables, antibiotic substance, antifungal substance, and hair tonic.

### EXAMPLES

The present invention will now be more particularly illustrated by the way of the following non-limiting examples (synthetic, testing and compounding examples). In the compounding examples, the compounding amount is given in terms of % by weight.

### Example 1

### Synthetic Example: Synthesis of Zinc Salt of PCA And Synthesis of Comparative Substances

DL-PCA zinc salt was synthesized according to the method in Japanese Patent Laid-Open No. 03/168,240. Specifically, DL-pyrrolidone carboxylic acid was reacted with zinc oxide in water at 100° C. for 2 hours and then stirred at room temperature for 5 hours, and the separated crystals were filtered. L-PCA zinc salt was synthesized as follows. In an autoclave, an aqueous solution of sodium L-glutamate monohydrate (61.1 g) was heated to 180° C. for two hours to obtain 50 wt % aqueous solution of sodium pyrrolidonecarboxylate. To 100.0 g (0.33 mol, pH 7.7, optical purity 84%, L/D ratio=92/8) of the 50 wt % aqueous solution of sodium pyrrolidonecarboxylate, 2.7 g of nitric acid (purity 60 wt %) was added to adjust to pH 5.2. 47.6 g (0.17 mol) of zinc sulfate 7-hydrate was dissolved in 34.2 g of water and the resulting solution was added to the aqueous sodium pyrrolidonecarboxylate solution (pH 4.1). This was stirred for 30 min at room temperature (pH 3.7) to obtain crystals, and then filtered. The resulting crystals were washed with water (21.9 g) to obtain 32.0 g (0.09 mol, yield 55%) of zinc pyrrolidonecarboxylate dihydrate. Optical purity was 99.8% (L/D ratio 99.9/0.1). A commercially available zinc salt of glycine (Tokyo Kasei) was used as a standard for comparison. Zinc laurate was synthesized by the following method. 250 mg (1.25 mmoles) of lauric acid was dissolved in 10 ml of ethyl alcohol and, also a 1% ethanolic solution of zinc acetate (Wako Pure Chemicals) was prepared. The 1% zinc acetate (15.1 ml; 0.69 mmole of zinc acetate; 0.55 equivalent to lauric acid) was added to 10 ml of the above-prepared ethanolic solution of lauric acid and the resulting precipitate was filtered. The resulting precipitate was washed with water, ethanol, and acetone and dried in vacuo to give 200 mg of zinc laurate.

Test Example 1: Action of PCA Zinc Salt On AP-1 Activation By Ultraviolet Ray

To human dermal fibroblasts which had reached confluence on an incubation plate, a test compound was added to such a concentration that does not damage fibroblasts, and the cells were incubated for 18 hours, and the incubated liquid was substituted with a phenol red-free medium. The fibroblasts were irradiated with ultraviolet rays (UVA: 20 J/cm²) using Dermaray M-DMR-80 (manufactured by Toshiba Iryoyohin). After 4 to 5 hours, the fibroblasts were recovered and the nucleoprotein was extracted therefrom by a conventional method. The resulting nucleoprotein was subjected to a gel shift assay or an ELISA assay (TransAM Kit, ACTIVE MOTIF) to detect activated AP-1. When applying the gel shift assay, the radioactive value of the AP-1 band was measured using a bio-imaging analyzer BAS 2000 (manufactured by Fuji Photo Film).

The suppression rate of the test compound for AP-1 activation was calculated from the following formula.
Suppression Rate for AP-1 Activation (%)={1−(A1−A3)/(A2−A3)}×100
A1: radioactive value of AP-1 band when a test compound was added
A2: radioactive value of AP-1 band when no test compound was added
A3: radioactive value of AP-1 band when no test compound was added and no irradiation with ultraviolet rays

An ELISA assay was performed in accordance with the manual of the kit used, and the activation of AP-1 was obtained by determination of the quantity of c-Jun or c-Fos.

The suppression rate of AP-1 (c-Jun or c-Fos) activation of a test compound was calculated from the following formula.
Suppression Rate for AP-1 Activation (%)={1−{B1−B3)/(B2−B3)}×100
B1: 450 nm absorbance when a test compound was added (per nucleoprotein)
B2: 450 nm absorbance when no test compound was added (per nucleoprotein)
B3: 450 nm absorbance when no test compound was added and no ultraviolet ray was irradiated (per nucleoprotein)

Results are shown in Table 1-3. The DL-PCA zinc salt and L-PCA zinc salt composition showed a high suppression rate as compared with zinc salt of fatty acid such as lauric acid (e.g., WO 00/44341) and zinc salt of amino acid such as glycine (e.g., WO 93/14748). The latter is known to have an ultraviolet preventing action due to induction of metallothionein of the skin. Such a result shows that the compound of the present invention is able to greatly suppress inflammation. Other known compounds, such as amino acid zinc and fatty acid zinc which have been known to have a protective action for the skin against ultraviolet ray, do not show such a result.

TABLE 1

| | Evaluated | |
|---|---|---|
| Test Compound | Concentration (μM) | Suppressing Rate (%) |
| Zinc | 50 | 38.8 |
| DL-Pyrrolidonecarboxylate | | |

TABLE 2

| | Evaluated | | |
|---|---|---|---|
| | Concen- | Suppressing | Suppressing |
| | tration | Rate (%) | Rate (%) |
| Test Compound | (μM) | (c-Jun) | (c-FOS) |
| Zinc L-Pyrrolidonecarboxylate | 10 | 109.5 | 69.3 |
| Zinc L-Pyrrolidonecarboxylate | 100 | 106.7 | 77.0 |

TABLE 3

| Test Compound | Evaluated Concentration (μM) | Suppressing Rate (%) |
|---|---|---|
| Zinc Salt of | 50 | −72.8 |
| Glycine | | |
| Zinc Laurate | 50 | 9.1 |

Compounding examples 1 to 13 are shown as follows. The preparations were prepared according to a conventional method. Compounding amounts were given in % by weight.

Compounding Example 1: Ointment

| | |
|---|---|
| L-PCA zinc salt | 1.0% |
| Benzalkonium chloride | 0.1% |
| Urea | 20.0% |
| White vaseline | 15.0% |
| Light liquid paraffin | 6.0% |
| Cetanol | 3.0% |
| Stearyl alcohol | 3.0% |
| Glyceryl monostearate | 5.0% |
| Perfume | q.s. |
| Antiseptic agent | q.s. |
| Buffer | 1.0% |
| Pure water | balance |

Compounding Example 2: Toilet Water

| | |
|---|---|
| L-PCA zinc salt | 3.0% |
| Glycolic acid | 5.0% |
| Glycerol | 3.0% |
| Sorbitol | 2.0% |
| Polyoxyethylene (20) oleyl ether | 1.0% |
| Ethanol | 15.0% |
| Zinc p-phenolsulfonate | 0.2% |
| Buffer | 0.1% |
| Perfume | 0.2% |
| Antiseptic agent | q.s. |
| Pure water | balance |

Compounding Example 3: Toilet Water

| | |
|---|---|
| DL-PCA zinc salt | 0.5% |
| Citric acid | 1.0% |
| Urea | 4.0% |
| Salicylic acid | 2.0% |
| Lactic acid | 2.0% |
| Glycerol | 2.0% |
| Betaine | 2.0% |
| Hyaluronic acid | 0.1% |
| Ethanol | 15.0% |
| Buffer | 0.1% |
| Perfume | 0.2% |
| Antiseptic agent | q.s. |
| Pure water | balance |

Compounding Example 4: Lotion

| | |
|---|---|
| L-PCA zinc salt | 0.5% |
| Lactic acid | 0.1% |
| Fruit acid | 0.1% |
| Glycerol | 4.0% |
| Kaolin | 1.0% |
| Calamine | 0.7% |
| Camphor | 0.2% |
| Ethanol | 14.0% |
| Perfume | q.s. |
| Pure water | balance |

Compounding Example 5: Cream

| | |
|---|---|
| L-PCA zinc salt | 1.0% |
| Resorcinol | 0.1% |
| Kojic acid | 1.0% |
| Stearic acid | 2.0% |
| Polyoxyethylene (25) cetyl ether | 3.0% |
| Glyceryl monostearate | 2.0% |
| Octyldodecanol | 10.0% |
| Cetanol | 6.0% |
| Reduced lanoline | 4.0% |
| Squalane | 9.0% |
| 1,3-Butylene glycol | 6.0% |
| Polyethylene glycol (1500) | 4.0% |
| Antiseptic agent | q.s. |
| Perfume | q.s. |
| Pure water | balance |

Compounding Example 6: Cream

| | |
|---|---|
| DL-PCA zinc salt | 1.0% |
| Glycolic acid | 2.0% |
| Solid paraffin | 5.0% |
| Beeswax | 10.0% |
| Vaseline | 15.0% |
| Liquid paraffin | 41.0% |
| 1,3-Butylene glycol | 4.0% |
| Glycerol monostearate | 2.0% |
| Polyoxyethylene (20) sorbitan monolaurate | 2.0% |
| Borax | 0.2% |
| Antiseptic agent | q.s. |
| Perfume | q.s. |
| Antioxidant | q.s. |
| Pure water | balance |

Compounding Example 7: Milky Lotion

| | |
|---|---|
| L-PCA zinc salt | 1.0% |
| Lactic acid | 2.0% |
| Stearyl alcohol | 0.5% |
| Hydrogenated palm oil | 3.0% |
| Liquid paraffin | 35.0% |
| Dipropylene glycol | 6.0% |
| Polyethylene glycol (400) | 4.0% |
| Sorbitan sesquioleate | 1.6% |
| Polyoxyethylene (20) oleyl ether | 2.4% |
| Carboxyvinyl polymer | 1.5% |
| Potassium hydroxide | 0.1% |
| Chelating agent | q.s. |
| Antiseptic agent | q.s. |
| Perfume | q.s. |
| Pure water | balance |

Compounding Example 8: Beauty Foundation

| | |
|---|---|
| L-PCA zinc salt | 0.5% |
| Fruit acid | 0.5% |
| Dipropylene glycol | 5.0% |
| Polyethylene glycol (400) | 5.0% |
| Ethanol | 10.0% |
| Carboxyvinyl polymer | 0.5% |
| Sodium alginate | 0.5% |
| Potassium hydroxide | 0.2% |
| Poloxyethylene (20) sorbitan monostearate | 1.0% |
| Sorbitol monooleate | 0.5% |
| Oleyl alcohol | 0.5% |
| Placenta extract | 0.2% |
| dl-α-Tocopherol acetate | 0.2% |
| Perfume | q.s. |
| Antiseptic agent | q.s. |
| Fading preventer | q.s. |
| Pure water | balance |

Compounding Example 9: Pack

| | |
|---|---|
| DL-PCA zinc salt | 3.0% |
| Isopropanol | 2.0% |
| Polyvinyl alcohol | 15.0% |
| Carboxymethyl cellulose | 5.0% |
| 1,3-Butylene glycol | 5.0% |
| Ethanol | 12.0% |
| Polyoxyethylene (20) oleyl ether | 0.5% |
| Perfume | q.s. |
| Antiseptic agent | q.s. |
| Buffer | q.s. |
| Pure water | balance |

Compounding Example 10: Foundation

| | |
|---|---|
| DL-PCA zinc salt | 5.0% |
| Salicylic acid | 0.5% |
| Liquid paraffin | 10.0% |
| Polyoxyethylene (20) sorbitan monooleate | 3.5% |
| Propylene glycol | 3.0% |
| Titanium oxide | 9.0% |
| Kaolin | 24.0% |
| Talc | 42.0% |
| Coloring pigment | 3.0% |
| Perfume | q.s. |
| Antiseptic agent | q.s. |
| Antioxidant | q.s. |

Compounding Example 11: Liquid Hand Soap

| | |
|---|---|
| L-PCA zinc salt | 5.0% |
| Sodium laurylsulfate | 30.0% |
| Betaine | 3.0% |
| Glycerol fatty acid ester | 1.0% |
| Phenoxyethanol | 1.0% |
| EDTA | 0.1% |
| Pure water | balance |

Compounding Example 12: Shampoo

| | |
|---|---|
| DL-PCA zinc salt | 0.1% |
| Polyoxyethylene (3) lauryl ether | |
| triethanolamine sulfate | 3.0% |
| Polyoxyethylene (3) lauryl ether sodium sulfate | 6.0% |
| Sodium laurylsulfate | 1.5% |
| Diethanolamide laurate | 3.0% |
| Betaine lauryldimethylaminoacetate | 2.5% |
| Cationized cellulose | 0.2% |
| Ethylene glycol distearate | 2.0% |
| Perfume | q.s. |
| Antiseptic agent | q.s. |
| Chelating agent | q.s. |
| Buffer | q.s. |
| Pure water | balance |

Compounding Example 13: Toilet Water For Hair

| | |
|---|---|
| DL-PCA zinc salt | 1.0% |
| Lactic acid | 0.02% |
| Oleyl alcohol | 0.2% |
| Liquid paraffin | 0.5% |
| Ethanol | 5.0% |
| Sorbitol | 4.0% |
| Polyoxyethylene (20) lauryl ether | 2.5% |
| Sorbitan monolaurate | 0.5% |
| Dye | 0.1% |
| Antiseptic agent | 0.1% |
| Perfume | 0.1% |
| Pure water | balance |

### INDUSTRIAL APPLICABILITY

The present invention is applicable in the cosmetic field.

While the invention has been described in detail with reference to preferred embodiments thereof, it will be apparent to one skilled in the art that various changes can be made, and equivalents employed, without departing from the scope of the invention. Each of the aforementioned documents, including the foreign priority document 2004-169655, is incorporated by reference herein in its entirety.

1. A method for treating or preventing inflammation comprising administering a composition comprising zinc pyrrolidonecarboxylate.

2. The method of claim 1, wherein said inflammation is of the skin.

3. The method of claim 2, wherein said inflammation is promoted by UV radiation.

4. The method of claim 2, wherein said inflammation is caused by skin damage.

5. The method of claim 2, wherein said inflammation causes aging of the skin.

6. The method of claim 5, wherein said aging is promoted by UV radiation.

7. The method of claim 2, wherein said inflammation is caused by hypersensitivity to sunlight.

8. The method of claim 2, wherein said inflammation is caused by an allergic reaction to light.

9. The method of claim 2, wherein said inflammation is caused by optical immunosuppression.

10. The method of claim 2, wherein said inflammation is caused by chapping of the skin.

11. The method of claim 10, wherein said chapping is caused by a wound or cracking of the skin.

12. The method of claim 2, wherein said inflammation is caused by a skin disease.

13. The method of claim 12, wherein said disease is selected from the group consisting of acne, keratosis, xeroderma, ichthyosis, and psoriasis.

14. The method of claim 1, wherein said zinc pyrrolidonecarboxylate is either in the L- or DL-configuration.

15. The method of claim 1, wherein said composition is a selected from the group consisting of an oil, lotion, cream, foundation, gel, shampoo, hair rinse, hair conditioner, enamel, lipstick, face powder, ointment, tablet, injection, granule, capsule, perfume, powder, cologne, toothpaste, soap, cleansing foam, bath salt, hair tonic, and sunscreen.
